(19) United States
(12) Patent Application Publication
(10) Pub. No.: US 2014/0276077 A1
(43) Pub. Date: Sep. 18, 2014

(54) ATTACHABLE ADAPTOR WITH CAVITY FOR ULTRASOUND DEVICE

(71) Applicant: G. Chad MORGAN, Chattanooga, TN (US)

(72) Inventor: G. Chad MORGAN, Chattanooga, TN (US)

(21) Appl. No.: 14/212,060

(22) Filed: Mar. 14, 2014

Related U.S. Application Data
(60) Provisional application No. 61/793,428, filed on Mar. 15, 2013.
(60) Provisional application No. 61/919,233, filed on Dec. 20, 2013.

(51) Int. Cl. A61B 8/00

(52) U.S. Cl. CPC A61B 8/4281; A61B 8/4455
USPC 600/459

(57) ABSTRACT

Embodiments associated with adaptors for ultrasound devices are described. In one embodiment, an adaptor includes a housing configured to attach to a head of the ultrasound device. The adaptor includes a cavity formed by the housing that is configured to receive and contain an ultrasound conductive medium that is preconfigured into a solidified form.

### CROSS REFERENCE TO RELATED APPLICATIONS

This patent disclosure claims the benefit of U.S. Provisional Patent Application Ser. No. 61/793,428 entitled “Attachable Adaptor with Recessed Cavity for Ultrasound Device,” filed on Mar. 15, 2013, and U.S. Provisional Patent Application Ser. No. 61/919,233 entitled “Gel with locking element and gel adaptor” filed on Dec. 20, 2013, which are all hereby wholly incorporated by reference in their entirety.

### BACKGROUND

Ultrasound devices operate with frequencies from 20 kHz up to several gigahertz. Ultrasound is a method of stimulating the tissue beneath the skin's surface using very high frequency sound waves and/or to provide imaging of internal structures.

Ultrasound is applied using an ultrasound device that includes a transducer or applicator that is in contact with a patient's skin. Gel is used on all surfaces of the device's head and on the patient to reduce friction and assist transmission of the ultrasonic waves. The gel (which is in liquid form) is typically squeezed out of a bottle and spread over the patient's skin. Liquid gel is difficult to contain within a desired area of the skin and the thickness of the gel cannot be controlled. Lack of a consistent and desired thickness of the gel leads to a less than optimal ultrasound application. When the ultrasound procedure is completed, the patient is required to wipe off the gel from the patient's skin. Typically, the gel is not completely removed and the cleaning process is uncomfortable.

### SUMMARY OF THE DISCLOSURE

In one embodiment of the disclosure, an adaptor for an ultrasound device is disclosed. The adaptor comprises a housing configured to attach to a head of the ultrasound device; and a cavity formed by the housing that is configured to receive and contain an ultrasound conductive medium that is preconfigured into a solidified form. In another embodiment, the adaptor is a retrofit component for attachment to the ultrasound device.

In another embodiment, the adaptor housing includes sidewalls configured as a cap structure having a first end and a second end that are both open, wherein the first end includes the cavity for containing the ultrasound conductive medium, and the second end is configured to join with and attach over the head of the ultrasound device.

In another embodiment, the adaptor housing includes a cavity sidewall that defines sides of the cavity, wherein a height of the cavity sidewall is less than a thickness of the solidified form of the ultrasound conductive medium.

In another embodiment, the housing is configured to attach to the head of the ultrasound device and be positioned between the head and the ultrasound conductive medium, wherein the cavity includes the solidified form of the ultrasound conductive medium inserted therein.

In another embodiment, the ultrasound device is an operational ultrasound applicator including an ultrasound transducer and including a generally flat applicator surface adjacent the ultrasound transducer that is configured to function with liquid gel; and wherein the adaptor is configured to convert the flat applicator surface of the ultrasound device to a surface with the cavity that is configured to hold the solidified form of the ultrasound conductive medium.

In one embodiment, the adaptor includes a recess for connecting with a protruding portion of the ultrasound conductive medium.

In another embodiment of the disclosure, an adaptor for an ultrasound device is disclosed wherein the ultrasound device is configured in an existing configuration that has a head with a generally flat applicator surface that operates with liquid gel. The adaptor comprises a housing configured to be attachable to the head of the ultrasound device, wherein the housing is configured to convert the ultrasound device from the existing configuration to a different configuration wherein the head can hold a preconfigured ultrasound conductive medium that is in a solidified form.

In another embodiment, the housing comprises sidewalls formed in a cap structure having a first end and a second end, wherein the first end is formed to mate with a shape of the head of the ultrasound device, and the second end defines a cavity configured to receive and contain the preconfigured ultrasound conductive medium on the applicator surface.

In another embodiment, the adaptor housing includes a cavity sidewall that defines sides of the cavity, wherein a height of the cavity sidewall is less than a thickness of the solidified form of the ultrasound conductive medium.

In one embodiment, the adaptor is a retrofit component for attachment to the head of the ultrasound device.

In another embodiment, the adaptor is an attachable and detachable component for the ultrasound device.

In another embodiment of the disclosure, a preconfigured ultrasound conductive medium is disclosed that comprises a solidified form of an ultrasound conductive medium, wherein the solidified form is preconfigured to mate with a receptacle of a retrofitting adaptor that is configured to attach to a head of an ultrasound device.

In another embodiment, the retrofitting adaptor comprises a housing configured to attach to the head of the ultrasound device; and wherein the receptacle is formed by the housing to be adjacent the head of the ultrasound device when the retrofitting adaptor is attached to the ultrasound device.

In another embodiment, the receptacle includes a receptacle shape and wherein the solidified form of the ultrasound conductive medium has a shape that generally corresponds to the receptacle shape to allow insertion within the receptacle.

In another embodiment, the solidified form includes a thickness that is greater than a height of the receptacle in the retrofitting adaptor.

In another embodiment of the disclosure, an adaptor for an ultrasound device is disclosed. The adaptor comprises a housing configured to attach to a head of the ultrasound device; a cavity formed by the housing and configured to be adjacent the head of the ultrasound device when the adaptor is attached to the head; and an ultrasound conductive medium contained within the cavity, wherein the ultrasound conductive medium is preconfigured into a solidified form.

In one embodiment, the adaptor is a retrofit component and includes the ultrasound device inserted into one end of the housing.

In another embodiment, the solidified form of the ultrasound conductive medium includes a shape configured to correspond to and mate with a shape of the cavity of the adaptor.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1A illustrates one embodiment of an ultrasound device shown with components unassembled including in embodiment of an adaptor.

FIG. 1B illustrates the adaptor of FIG. 1A attached to the ultrasound device in an assembled view.

FIG. 2A illustrates a top view of one embodiment of the adaptor.

FIG. 2B illustrates a side view of one embodiment of the adaptor.

FIG. 2C illustrates a perspective view of one embodiment of the adaptor.

FIGS. 3A, 3B, and 3C illustrate top, perspective, and side views of another embodiment of a preconfigured gel configured with a locking ring.

FIGS. 4A, 4B, 4C, and 4D illustrate a top view, left side view, right side view, and bottom view, respectively, of another embodiment of an adaptor including a locking recess.

FIG. 4E illustrates a cross-section view of the adaptor through section A-A of FIG. 4B.

### DETAILED DESCRIPTION

Various embodiments of an attachable adaptor are disclosed herein for an ultrasound device and/or imaging device. In one embodiment, the attachable adaptor is configured to attach to an applicator tip/head or diaphragm of an ultrasound device. Once attached, the adaptor converts a flat surface of the diaphragm (e.g., the applicator surface that is placed against the skin of a patient) to a surface that includes a gel cavity/receptacle. The cavity is configured to receive and maintain a portion of a preconfigured conductive medium (e.g., ultrasound gel in a solidified state) that has a preconfigured shape to fit into the cavity. Thus the adaptor converts an ultrasound device to one that can self-contain ultrasound gel. In one embodiment, the adaptor is a retrofit component for attachment to an existing ultrasound device/applicator.

With reference to FIGS. 1A and 1B, one embodiment of an ultrasound device 100 is shown in a partially unassembled state (FIG. 1A) and in an assembled state (FIG. 1B). The device 100 is configured as a hand-held device including a handle 105 that may include zero or more finger grips 110 (e.g., indentations, ridges, and so on). The handle 105 is connected to a head 115 that includes one or more sides that connect to or include a diaphragm 120 that contains one or more ultrasound transducers therein (not shown). The head 115 and diaphragm 120 are sometimes referred to as simply the “head,” “nose,” or “transducer.”

In the illustrated embodiment, the diaphragm 120 includes a generally flat applicator surface 125, which is the surface that is put in contact with a target (e.g., ultrasound patient). The applicator surface 125 may have a variety of shapes depending on the designed shape of the head 115. The shape may be circular, oval, elliptical, rectangular, variations of these shapes, or other implemented shape. In some embodiments, the applicator surface 125 may be straight or curved (e.g., convex shape) from one side to the other.

In one embodiment, diaphragm 120 is configured with a connector (not shown) that is threaded to insert and connect with a corresponding threaded socket in the head 115. In another embodiment, the connector may be configured as a quick-connect/disconnect device so that the diaphragm 120 can be connected to the head 115 by pushing and snapping into place or disconnected by pulling off with a small amount of force.

Thus, in one embodiment, the diaphragm 120 is configured as a removable and replaceable component. In another embodiment, the head 115 and diaphragm 120 are integral with each other. In general, the diaphragm 120 is the portion that contains a transducer (e.g., piezoelectric crystal disposed within the diaphragm) that generates ultrasound energy. Of course, any form of ultrasound device including a device with multiple transducers can be used.

With further reference to FIG. 1A, in one embodiment, an attachable adaptor 135 is shown that is configured to convert the flat surface 125 of the ultrasound device 100 to have a receptacle/cavity 145 for receiving and holding a preconfigured ultrasound conductive medium 140 (e.g., a piece of preconfigured solidified gel). In other words, the receptacle/cavity 145 is a defined containment area for the preconfigured gel 140.

For example, the adaptor 135 is configured to attach to the head portion 115/120 of the device 100 and convert the head portion 115/120 from an existing configuration (e.g., first state having a flat surface) that does not have a gel receiving cavity to a different configuration (e.g., second state that is a non-flat surface) that includes a gel receiving cavity. In one embodiment, the housing of the adaptor 135 is configured to attach to the head/diaphragm 115/120 and be positioned between the head/diaphragm and the ultrasound conductive medium 140. In one embodiment, the components are in a stacked relationship, for example, (1) the ultrasound device, (2) the adaptor, and (3) the preconfigured ultrasound conductive medium.

After the adaptor 135 is attached, the ultrasound device 100 no longer has a flat applicator surface 125 that uses liquid gel during an ultrasound procedure. Rather, the preconfigured conductive medium 140 is inserted into the adaptor 135, which holds the preconfigured conductive medium 140 in place. The preconfigured conductive medium 140 provides for a predetermined, consistent, and controlled amount/thickness of ultrasound conductive medium as compared to the random, uncontrolled amounts of liquid gel that are used.

In one embodiment, the adaptor 135 is configured as a cap structure so that it can be clipped-on, snapped-on, slid-on, attached to, or otherwise connected over the head/diaphragm 120 by pushing on and/or snapping into place. The adaptor 135 may also be disconnected by pulling off with a small amount of force. Thus in one embodiment, the adaptor 135 is an attachable and detachable component.

The shape and size of the adaptor 135 is configured to correspond to the configuration of the head/diaphragm 120 of a targeted ultrasound device for which the adaptor 135 is designed to mate with. Accordingly, a custom fitted adaptor 135 can be formed for different types and models of ultrasound transducers or probes.

FIG. 1B shows the diaphragm 120 inserted into the adaptor 135, or in other words, the adaptor 135 attached to the diaphragm 120. The adaptor 135 may be configured to be held in place by at least surface tension or pressure with surfaces of the diaphragm 120, by friction with the inside surfaces of the adaptor 135, by an adhesive, by mating with corresponding lips or edges from the adaptor 135, and/or from the head 115/120, and/or by a connecting device.

The adaptor 135 is configured with edges or side walls that define a cavity 145 that is configured to receive and contain an ultrasound conductive medium 140 used during an ultrasound or imaging scan. In one embodiment, the ultrasound conductive medium 140 is a portion of ultrasound gel that is preconfigured to fit into the cavity 145. The preconfigured gel 140 is configured with a shape to correspond to and/or mate with the shape of the gel cavity 145. As seen in FIG. 1B, the gel 140 is inserted into the cavity 145 of the adaptor 135.

The gel 140 and adaptor 135 are configured in a cooperative relationship such that the gel 140 extends a distance beyond the edges of the adaptor 135 once the gel 140 is inserted. For example, the gel 140 includes a thickness that is greater than the sidewalls of the cavity 145. Thus, the gel 140 is positioned to provide an interface and coupling medium between the applicator surface 125 of the device 100 and a patient's skin where the gel 140 can contact the skin.

In one embodiment, the adaptor 135 holds the preconfigured gel 140 so that the preconfigured gel 140 is disposed directly on the applicator surface 125 of the diaphragm 120. The preconfigured gel 140 is formed to generally correspond to the shape of the receptacle/cavity 145 of the adaptor 135 so the gel 140 fits into the cavity 145. Thus in one embodiment, the adaptor 135 includes two cavities: one for receiving the ultrasound device diaphragm 120 (shown as cavity 215 in FIGS. 2B and 2C) and one for receiving the preconfigured gel 140 (cavity 145). The adaptor 135 is explained in more detail with reference to FIGS. 2A-2C.

With reference to FIGS. 2A-C, one embodiment of the adaptor 135 is shown in a top view in FIG. 2A, a side view in FIG. 2B, and a perspective view in FIG. 2C along with the preconfigured gel 140.

In one embodiment, the adaptor 135 includes a housing formed in a generally cap-like structure that can fit over the head 115 of the ultrasound device 100 and over the applicator surface 125. In another embodiment, the adaptor 135 has generally a tube structure.

The adaptor housing is made from a material that can operate with ultrasound energy generated from the transducer of the ultrasound device 100. The material may be metal, metallic, polymer, plastic, or other material that functions with ultrasound energy so as to have minimal disruptive or interference effects from the intended operation of the ultrasound device. In another embodiment, the adaptor 135 is made using material that can act as an insulator of ultrasound energy so as to function as a directional control component to direct ultrasound energy from the ultrasound device to be transmitted out from the cavity 145 of the adaptor 135.

With reference to FIG. 2A, the adaptor 135 is configured with a circular shape defined by an exterior sidewall 210 that forms the housing. The adaptor 135 is circular since the targeted ultrasound device 100 has a circular head 115. Of course the shape of the adaptor 135 will be different for a differently shaped ultrasound device.

When connected to the ultrasound device 100, a cavity sidewall 220 at one end of the sidewall 210 is configured to extend out from the applicator surface 125 (see FIG. 2B, FIGS. 1A-1B) and define the gel receiving cavity 145. The gel cavity 145 has a perimeter defined by the cavity sidewall 220 on a first end of the adaptor 135 (e.g., gel side).

In one embodiment, the housing of the adaptor 135 includes an inside surface 205 that defines an edge or stop. When the adaptor 135 is attached to the ultrasound device 100, the diaphragm 120 of the ultrasound device is inserted through an opposite second end of the adaptor that defines an opening 215. The diaphragm 120 would slide into the adaptor 135 and contact against the surface 205, which would stop the diaphragm 120 from sliding through the entire opening of the adaptor 135. Of course, other configurations of a stop mechanism can be implemented (e.g., internal ribs or edges, etc.). In another embodiment, the housing is configured as a tube housing that tapers toward one end that functions as the stop mechanism. Thus the narrowing of the tube causes contact with the ultrasound head thereby locking the adaptor 135 in place (e.g., tight fit). Accordingly, the inside surface of the housing of the adaptor 135 does not have an inner stop edge.

Once inserted into the adaptor 135, the exterior applicator surface 125 of the diaphragm (see FIG. 1B) is exposed in the gel cavity 145 and forms the bottom surface of the cavity 145. Thus, when the preconfigured piece of gel 140 is inserted into the cavity 145, the preconfigured gel 140 lays against the applicator surface 125 of the diaphragm 120. The cavity sidewall 220 functions to hold the preconfigured gel 140 from moving or falling off the ultrasound device.

In one embodiment, the height of the cavity sidewall 220 is less than the thickness of the preconfigured gel 140. In this manner, the top exposed surface 150 of the gel 140 extends beyond the housing of the adaptor 135 when the gel 140 is inserted in the cavity 145 in order to contact an object of interest (e.g., skin) (see FIG. 2C and FIG. 1B).

In another embodiment, the inside surface 205 may be a dividing wall that extends across the adaptor 135. Thus the entire wall acts as a stop mechanism. In this manner, the dividing wall 205 would separate the preconfigured gel 140 in the cavity 145 and the applicator surface 125 of the diaphragm that is inserted into the adaptor 135. Thus the applicator surface 125 would not be exposed and would not directly contact the preconfigured gel 140.

The opening 215 (e.g., the second cavity in the adaptor 135) is configured to fit on (attach to) and join with or connect to the ultrasound device. As previously stated, the adaptor 135 is shown in a generally circular shape for purposes of example only, but other shapes may be implemented based on the shape of the ultrasound device for which the adaptor 135 is configured to fit on (e.g., rectangular, oval, other polygon shape, irregular shape, and so on). In general, once attached to an ultrasound device, the adaptor 135 converts the flat applicator surface 125 of the ultrasound device to a surface that has side walls 220 extending out from the applicator surface 125. The side walls 220 are configured to can contain a preconfigured ultrasound gel and hold the gel in place during an ultrasound procedure (e.g., during therapy, imaging, etc.). In this manner, liquid gel is not needed to be spread over the skin of a patient.

In one embodiment, the sidewalls 210 and 220 are a continuous edge or rim around the perimeter of the cavity 145. In another embodiment, the sidewall 210 and 220 may include one or more notches (not shown). A notch may be used to remove the preconfigured gel 140 from within the cavity 145 by inserting a finger in the notch to access the gel 140 within the cavity 145. In another embodiment, the cavity sidewall 220 may be perforated or be configured as two or more portions such as prongs that can hold a piece of solidified gel.

With reference again to FIG. 1A, in one embodiment, in the inside of the diaphragm 120, the diaphragm 120 includes one or more transducers (e.g., piezoelectric crystal) (not shown) for generating ultrasound waves. The transducer is connected within the diaphragm 120 and secured against an inside surface of the diaphragm 120.

The various dimensions shown are only exemplary of one embodiment. It is not intended to limit the construction of the adaptor 135 shown since the adaptor 135 can be formed with different shapes and sizes.

Ultrasound Device Components

With reference again to FIG. 1A, in one embodiment, the handle 105 and head 115 are formed from a housing that contains one more components (not shown) configured to generate and/or detect ultrasound energy. In one embodiment, the ultrasound device 100 includes an energy generating module operative to generate a driving signal that can be transformed into ultrasonic energy. The energy generating module includes a local power source or receives power from a remote source via a power cord, an oscillator, and a driver component. The portable ultrasound device 100 also includes an ultrasound transducer having a piezoelectric component, which is disposed within the diaphragm 120 and is generally near or adjacent to the applicator surface 125. The ultrasound transducer is operative to receive the driving signal from the energy generating module and transform the driving signal into ultrasonic energy. There are many different types of internal components that can be used to implement the ultrasound device 100. Since they are not the focus of the present disclosure, they are not described in detail.

In another embodiment, the device 100 may include an internal memory for storing ultrasound data collected by the device 100. The device 100 may include an interface for communicating the data from the memory to a remote device. The device 100 can be configured to communicate the data via a wire connection and/or a wireless connection.

Preconfigured Ultrasound Conductive Medium

With reference to FIG. 2C, in one embodiment, the preconfigured gel 140 is an ultrasound conductive medium for use with therapeutic or imaging ultrasounds and electrotherapy devices. In one embodiment, the preconfigured gel 140 is formed or molded into a desired shape that fits into and is contained within the cavity 145 of the adaptor 135 for which the gel is made. For example, the preconfigured gel 140 is a solidified form of an ultrasound conductive medium where the solidified form is preconfigured to mate with the receptacle/cavity 145 of the retrofitting adaptor 135, which is configured to attach to the head of an ultrasound device.

In one embodiment, a process to form the preconfigured gel 140 may involve using molds. Multiple molds can be used to create multiple pieces of the preconfigured gel at a time. For example, a tray of patterned shapes can be used where a gel composition in a liquid or aqueous form is filled into each patterned shape. The liquid gel composition is then processed to solidify the gel to a desired extent so that its shape is set (e.g., the solidified composition holds its shape, maintains its dimensions, does not flow, and/or does not take the shape of its container when placed in the container).

The solidifying process may involve curing, heating, cooling, or other process to solidify the aqueous composition. In different embodiments, the preconfigured gel 140 can be solidified to different degrees or ranges as desired such as being a soft and flexible object, being a rigid object, or any state in between (e.g., semi-rigid, elastic, and/or flexible structure). Of course, other processes may be used to create the preconfigured gel such as injection molding, 3-D printing, and so on.

In one embodiment, the preconfigured gel 140 is maintained within the gel cavity 145 of the adaptor 135. Thus during a scan, the gel 140 moves with the ultrasonic device 100 by being a part of the adaptor 135. The top exposed surface 150 of the preconfigured gel 140 is in contact with a patient's skin and acts as a lubricant to help the ultrasound device 100 slide across the skin from area to area.

Air and other gases may impede sound waves. Thus, the solidified gel 140 functions to prevent the formation of air bubbles between the transducer and the patient's skin and helps conduct sound waves from the transducer into the patient's body. Spreading unknown amounts of liquid gel on a patient may be eliminated.

The preconfigured piece of gel 140 maintains a predetermined thickness of ultrasound conductive medium. The preconfigured gel 140 thus provides a consistent and controlled thickness of the ultrasound conductive medium between a transducer of the ultrasound device and a patient. The consistent and controlled thickness of gel may improve the quality and/or consistency of the ultrasound energy applied to a patient because the thickness of the solidified gel 140 does not change. Thus the transducer and the object of interest (e.g., skin) are maintained at a consistent distance from each other.

After being used in an ultrasound procedure, the preconfigured gel 140 may be removed from the adaptor 135 and replaced with a new piece of preconfigured gel. Of course, the same piece of gel may be used for multiple procedures since it is solidified and moves with the adaptor. However, there may be issues with sterility from multiple uses that may not be desired.

Locking Mechanism Embodiment

FIGS. 3A, 3B, and 3C illustrate top, perspective, and side views, respectively of another embodiment of a preconfigured gel 300 configured with a locking element/mechanism. The locking element is generally a protruding portion of solidified gel that extends out from the gel surface or side wall to help restrict movement of the gel 300 when inserted into a gel cavity of an adaptor (e.g., like adaptor 135). In one embodiment, the side wall of the gel 300 is formed with a ring 305 that extends out from the sidewall and extends around the perimeter of the gel 300. From a different perspective, the ring 305 may be formed by having the base of the gel have a larger diameter that the top portion of the gel 300 as seen in the top view of FIG. 3A. The ring 300 may be positioned along any desired location along the height of the sidewall.

In other embodiments, rather than being one continuous ring, the locking ring 305 may be configured as one or more partial rings where each partial ring is less than the circumference of the gel 300. In another embodiment, the locking ring 305 may be configured as one or more ribs, lips, edges, or other protrusions that extend out from the sidewall in horizontal and/or vertical directions and may have any desired shape.

The ring 305 is configured to fit into a corresponding locking ring/recess in a gel adaptor for attachment to an ultrasound device. When the gel 300 is inserted into a gel cavity in the adaptor (e.g., in the adaptor 135 in FIG. 2C, in adaptor 400 in FIG. 4A, or similar cavity), the gel 300 is restricted from unintentionally falling out of the gel cavity.

In another embodiment, the gel 300 is configured in a predefined shape and includes indentations in one or more surfaces. For example, the indentations may be a word or phrase. As seen in FIGS. 3A and 3B, the gel 300 includes the words “Gel Shot” indented on the top surface.

With reference to FIGS. 4A-4E, one embodiment of a gel adaptor 400 is shown in a variety of views. For example, FIG. 4A: top perspective view; FIG. 4B: left side view; FIG. 4C: right side view; FIG. 4D: bottom perspective view; and FIG. 4E: cross-section view of section A-A from FIG. 4B.

The gel adaptor 400 is configured to attach to the head of an ultrasound device (as previously described) that does not have a gel cavity for holding a piece of gel preconfigured with a corresponding shape. Once attached, the adaptor 400 converts the existing flat applicator surface 125 of the ultrasound device 100 to a device with a gel cavity that can receive and hold a preconfigured piece of gel (e.g., gel 140—FIGS. 1A-1B; gel 300—FIGS. 3A-3C).

The adaptor 400 is configured with a top opening (gel cavity) 405 (FIG. 4A) and a bottom opening 410 (FIG. 4D). The housing of the adaptor 400 may be configured with a shape that corresponds to the shape of an ultrasound head so that the bottom opening 410 and sidewalls connect/attached to the ultrasound head. In that regard, the adaptor 400 may be configured with various internal shapes and edges 415 to contact surfaces of the ultrasound head for a better fit. In other embodiments, the internal portion of the adaptor 400 may include one or more lips 420 to provide additional connection points. The top opening/cavity 405 is configured to generally match the shape of the preconfigured gel being used (e.g., preconfigured gel 300 (FIG. 3A-3C)). Other shapes may include oval, rectangular, or other polygonal shape.

With reference to FIG. 4E, the adaptor 400 is configured with a gel locking ring/recess 425 within the gel cavity 405. The locking recess 425 generally corresponds to the locking ring 305 of the gel 300 and is configured to receive/connect with the locking ring 305. Since the solidified gel 300 is a flexible/malleable substance, the gel 300 can be pressed into the cavity 405 until the two shapes align (e.g., the protruding portion 305 of the gel is inserted into the recess 425). Thus, the locking ring 305 inserts into the corresponding locking recess 415 to lock the gel 300 in the cavity 405. Accordingly, the gel 300 is held in the cavity 405.

Overall, in one or more embodiments, the adaptor disclosed herein is a component for retrofitting an existing ultrasound device/applicator (e.g., an operational device that functions without the adaptor). The adaptor modifies an applicator surface of the ultrasound device to have a gel receiving cavity or receptacle. Thus existing ultrasound applicators that are functional can be modified rather than replacing the ultrasound applicator or system with a new system, which is more costly. Furthermore, the use of liquid gel can be eliminated as described herein.

### DEFINITIONS

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

The term “conductive medium” is used to refer to a substance that is used during an ultrasound procedure that assists in coupling the ultrasound device/probe head or applicator tip to a subject/target (e.g., the skin of a patient or other surface) and conducts ultrasound energy. Typically, the conductive medium is ultrasound gel but other substances can be used such as shampoo, hairstyling gel, hand lotion, hand sanitizer, liquid dishwashing detergent, olive oil (or other oil based substances), or other substance with a composition that is appropriate to function with an ultrasound device. Many substances can form gels when a suitable thickener or gelling agent is added to their formula to change the viscosity. These substances are preconfigured into a solidified state as an individual piece of conductive medium as explained previously (e.g., solidified state may be any state in which the gel maintains its shape and dimensions, and does not flow). References to the term “gel” is intended to refer to any of these conductive media that is appropriate for an ultrasound procedure.

While example devices, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this application is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

What is claimed is:

1. An adaptor for an ultrasound device, the adaptor comprising:
a housing configured to attach to a head of the ultrasound device; and
a cavity formed by the housing that is configured to receive and contain an ultrasound conductive medium that is preconfigured into a solidified form.

2. The adaptor of claim 1, wherein the adaptor is a retrofit component for attachment to the ultrasound device.

3. The adaptor of claim 1, wherein the housing includes sidewalls configured as a cap structure having a first end and a second end that are both open, wherein the first end includes the cavity for containing the ultrasound conductive medium, and the second end is configured to join with and attach over the head of the ultrasound device.

4. The adaptor of claim 1, wherein the housing includes a cavity sidewall that defines sides of the cavity, wherein a height of the cavity sidewall is less than a thickness of the solidified form of the ultrasound conductive medium.

5. The adaptor of claim 1, wherein the housing is configured to attach to the head of the ultrasound device and be positioned between the head and the ultrasound conductive medium, wherein the cavity includes the solidified form of the ultrasound conductive medium inserted therein.

6. The adaptor of claim 1, wherein the ultrasound device is an operational ultrasound applicator including an ultrasound transducer and including a generally flat applicator surface adjacent the ultrasound transducer that is configured to function with liquid gel; and
wherein the adaptor is configured to convert the flat applicator surface of the ultrasound device to a surface with the cavity that is configured to hold the solidified form of the ultrasound conductive medium.

7. The adaptor of claim 1, wherein the adaptor includes a recess for connecting with a protruding portion of the ultrasound conductive medium.

8. An adaptor for an ultrasound device, wherein the ultrasound device is configured in an existing configuration that has a head with a generally flat applicator surface that operates with liquid gel, the adaptor comprising:
a housing configured to be attachable to the head of the ultrasound device, wherein the housing is configured to convert the ultrasound device from the existing configuration to a different configuration wherein the head can hold a preconfigured ultrasound conductive medium that is in a solidified form.

9. The adaptor of claim 8, wherein the housing comprises sidewalls formed in a cap structure having a first end and a second end, wherein the first end is formed to mate with a shape of the head of the ultrasound device, and the second end defines a cavity configured to receive and contain the preconfigured ultrasound conductive medium on the applicator surface.

10. The adaptor of claim 8, wherein the housing includes a cavity sidewall that defines sides of the cavity, wherein a height of the cavity sidewall is less than a thickness of the solidified form of the ultrasound conductive medium.

11. The adaptor of claim 8, wherein the adaptor is a retrofit component for attachment to the head of the ultrasound device.

12. The adaptor of claim 8, wherein the adaptor includes a recess for connecting with a protruding portion of the ultrasound conductive medium.

13. The adaptor of claim 8, wherein the adaptor is an attachable and detachable component for the ultrasound device.

14. A preconfigured ultrasound conductive medium comprising:
a solidified form of an ultrasound conductive medium, wherein the solidified form is preconfigured to mate with a receptacle of a retrofitting adaptor that is configured to attach to a head of an ultrasound device.

15. The preconfigured ultrasound conductive medium of claim 14, wherein the retrofitting adaptor comprises:
a housing configured to attach to the head of the ultrasound device; and
wherein the receptacle is formed by the housing to be adjacent the head of the ultrasound device when the retrofitting adaptor is attached to the ultrasound device.

16. The preconfigured ultrasound conductive medium of claim 14, wherein the receptacle includes a receptacle shape and wherein the solidified form of the ultrasound conductive medium has a shape that generally corresponds to the receptacle shape to allow insertion within the receptacle.

17. The preconfigured ultrasound conductive medium of claim 14, wherein the solidified form includes a thickness that is greater than a height of the receptacle in the retrofitting adaptor.

18. An adaptor for an ultrasound device, the adaptor comprising:
a housing configured to attach to a head of the ultrasound device;
a cavity formed by the housing and configured to be adjacent the head of the ultrasound device when the adaptor is attached to the head; and
an ultrasound conductive medium contained within the cavity, wherein the ultrasound conductive medium is preconfigured into a solidified form.

19. The adaptor of claim 18, wherein the adaptor is a retrofit component and includes the ultrasound device inserted into one end of the housing.

20. The adaptor of claim 18, wherein the solidified form of the ultrasound conductive medium includes a shape configured to correspond to and mate with a shape of the cavity of the adaptor.
